(19) United States
(12) Patent Application Publication
(10) Pub. No.: US 2005/0158404 A1
(43) Pub. Date: Jul. 21, 2005

(54) Composition and method for treatment of acne

(76) Inventor: Dean R. Goodless, Celebration, FL (US)

Correspondence Address: Shannon McGarrah;Amin Law, LLC, Ste. 500, 217 N. Jefferson St., Chicago, IL, 60661, US

(21) Appl. No.: 10/991,262

(22) Filed: Nov. 17, 2004

Related U.S. Application Data
(60) Provisional application No. 60/523,169, filed on Nov. 18, 2003.

(51) Int. Cl. A61K 35/78; A61K 31/4745

(52) U.S. Cl. 424/725; 424/726; 514/283

(57) ABSTRACT

The present invention relates to a nutritional product, dietary supplement or pharmaceutical composition for the treatment of acne and related skin disorders. The invention also relates to a composition having a combination of ingredients such that it reduces symptoms associated with acne and related skin disorders. The invention relates to such a composition containing a variety of vitamins, minerals and one or more herbal medicine sources. In one preferred embodiment, the vitamins, minerals and one or more herbal medicine sources are present in an amount such that its mechanism of action is through inhibition of the growth of Propionobacterium acnes (P. acnes) and the inhibition of lipogenesis of sebum production. In another preferred embodiment, the invention contains a Vitamin A source to normalize keratinoid maturation, proliferation and turnover of skin cells. In another preferred embodiment, the invention further contains at least one of a Vitamin E source or Selenium. In another preferred embodiment, the invention further contains at least one of a Vitamin B₆ source, a Pantothenic Acid source, Zinc or any active salt, and Chromium or any active salt. In another preferred embodiment, the invention further contains any one or more of an herbal source of Berberis aquifolium (Oregon grape root), Barberis vulgaris (barberry), Hydrastis Canadensis (goldenseal), or other herbal source of the compound berberine. The present invention also relates to a method of administering a therapeutically effective amount of the combination of ingredients for the treatment of acne and related skin disorders.

### CROSS REFERENCE TO RELATED APPLICATIONS

This application claims the benefit of U.S. provisional application No. 60/523,169, filed on Nov. 18, 2003.

### FIELD OF INVENTION

The present invention relates to a composition for use in a nutritional product, dietary supplement or pharmaceutical composition wherein the composition provides a beneficial use in the treatment of acne and related skin disorders. The invention also relates to a composition having, in addition to the treatment of acne and related skin disorders, a combination of ingredients such that it reduces symptoms associated with acne and related skin disorders. Such symptoms include, but are not limited to, the reduction of redness, blemishes and oily skin associated with acne and other skin disorders. The present invention also relates to a method of administering a therapeutically effective amount of the combination of ingredients for the treatment of acne and related skin disorders.

### BACKGROUND OF THE INVENTION

Acne vulgaris is a common skin disorder affecting the majority of people at some time in their life, particularly during adolescence. The condition is often mild, but in some cases can cause permanent scarring and significant social and emotional consequences.

Acne is believed to be caused by the action of hormones on the skin's oil glands, otherwise known as sebaceous glands. It is an inflammatory condition that is primarily found on the face, neck, shoulders and back, attributed to the higher amount of sebaceous glands found in these areas. The production of oil by the sebaceous glands leads to plugged skin pores and outbreak of lesions. Acne lesions produced range from the most basic lesion known as a microcomedo, which is an enlarged and plugged hair follicle, to more severe lesions called papules, pustules (pimples), nodules and cysts. Cysts are painful lesions that can cause scarring and are classified as a type of acne known as cystic acne.

At the most basic level, pilosebaceous units consisting of a sebaceous gland connected to a follicle containing a fine hair are present under the skin's surface. During normal functioning, the sebaceous glands produce an oily substance known as sebum that drains onto the surface of the skin through an opening in the follicle called a pore. Also involved are keratinocytes, which are cells that line the follicle. These keratinocytes, along with the hair and sebum, may plug the follicle and prevent the sebum from reaching the surface of the skin as it would during normal functioning. This plug allows the bacteria Propionobacterium acnes (P. acnes) to grow in the plugged follicle. P. acnes normally grows on the surface of the skin and produces chemicals and enzymes which attract white blood cells. When P. acnes bacteria is present in the follicle, the white blood cells also enter the plugged follicle. When the wall of the plugged follicle is disrupted, the hair, sebum, bacteria and cells spill onto the skin, causing lesions or pimples.

Many programs are available for the treatment of acne vulgaris. Treatment programs usually involve a topical or a systemic (i.e. oral) treatment, used alone or in combination with one another. Common topical treatments include retinoic acid, benzoyl peroxide, resorcinol and salicylic acid. Side effects from these medications include dryness and irritation of the skin, redness or burning and allergic contact dermatitis. Other topical treatments for moderate to severe inflammatory acne involve the use of prescription antibiotics such as clindamycin, erythromycin and tetracycline. These antibiotics are used to prevent mild inflammatory acne but have little therapeutic effect with existing acne. Benzoyl peroxide, tretinoin, adapelene, and azelaic acid are other prescription medications used. Common side effects from these prescription medications include stinging, burning, redness, peeling or photosensitization of the skin.

Oral antibiotics such as clindamycin, erythromycin, tetracycline and ampicillin may be helpful in preventing new inflammation related to acne but can cause unwanted side effects including allergic reactions, gastrointestinal upset, interference with birth control pill effectiveness, photosensitization, and with longstanding use, bacterial antibiotic resistance.

Treatment for severe nodular or cystic acne may be given through a dermatologist with a prescription medicine such as isotretinoin, marketed by Roche Pharmaceuticals, Inc. as Accutane. Side effects of isotretinoin include dry eyes, mouth, lips, and skin; sensitivity to the sun; and itching. Isotretinoin also has the potential to cause birth defects in a developing fetus and has been linked to depression and suicide.

A number of studies have investigated the usefulness of various nutritional supplements for the treatment and amelioration of acne. Various vitamins and minerals such as Zinc, Vitamin A, Vitamin E and Pantothenic Acid have been studied to establish their effectiveness in treating acne and related conditions. In-vitro studies have examined the mechanisms whereby certain herbs may effect oil production by sebaceous glands and inhibit the growth of P. acnes, the bacteria thought to be a contributory cause of acne. Several examples of such herbs include Coptidis rhizome and Scutillaria species (Skullcap). The use and interest of herbal medicines has generally increased due to the common consideration and perception that these medicines are relatively safer than chemically synthesized drugs.

There is therefore a need and a demand for a beneficial compound used for the treatment of acne vulgaris that minimizes or eliminates the side effects such as redness, dryness, itching, sensitivity to the sun, etc., associated with current topical and oral treatments. There is also a need for a beneficial compound used for the treatment of acne that has a more effective therapeutic use in both preventing acne vulgaris and treating existing acne conditions. There is a need and a demand for such beneficial compound in which the composition is a natural product consisting of various vitamins and minerals as well as certain herbal medicines demonstrated to be effective in the intended treatment.

### SUMMARY OF THE INVENTION

The present invention relates to a nutritional product, dietary supplement or pharmaceutical composition for the treatment of acne and related skin disorders.

A general object of this invention is to provide a beneficial composition as a nutritional product, dietary supplement or pharmaceutical composition used for the treatment of acne and related skin disorders.

A further object of this invention is to provide such a composition used for the primary purpose for the treatment of acne and related skin disorders and additionally for use in the treatment of symptoms associated with acne and related skin disorders.

A more particularized object of this invention is to afford such treatment by providing a therapeutically effective amount of components to reduce the growth of P. acnes and sebum production, thereby offering reduction of symptoms of acne and related skin disorders such as redness, irritation, blemishes, and oiliness of the skin.

Goals of treatment using this invention include healing existing lesions, stopping new lesions from forming, preventing scarring and minimizing the psychological stress and embarrassment caused by acne.

The present invention represents a specific combination of several different vitamins, minerals and herbal ingredients to capitalize on any additive as well as any synergistic benefits of these compounds in the prevention or amelioration of acne breakouts and associated symptoms. The components of the composition include, without limitation, Vitamin A, Vitamin E, Vitamin B6, Pantothenic Acid, Zinc, Selenium, Chromium and any one or more of an herbal source of the compound berberine and other isoquinoline alkaloids.

The prior art has generally failed to provide a natural composition that is effective for the treatment of acne vulgaris and related skin conditions. The prior art has also failed to provide a beneficial compound that acts both to prevent acne and treat existing acne conditions. The prior art has also failed to provide a composition used for such treatment that reduces or eliminates possible sides effects such as redness, dryness, itching, burning or photosensitization.

### DETAILED DESCRIPTION OF THE INVENTION

The present invention provides a beneficial composition for the use in a nutritional product, dietary supplement or pharmaceutical composition that contains a combination of vitamins and minerals as well as one or more herbal ingredients. The present invention is intended for the use in the treatment of acne vulgaris and related skin disorders.

The composition generally contains, without limitation, Vitamin A, Vitamin E, Vitamin B6, Pantothenic Acid, Zinc, Selenium, Chromium and any one or more of an herbal source of Berberis aquifolium (Oregon grape root), Berberis vulgaris (barberry), Hydrastis Canadensis (goldenseal), or other herbal source of the compound berberine and other isoquinoline alkaloids in an effective therapeutic amount for the treatment of acne vulgaris and related skin disorders. The individual components may be included in the composition in any of their pharmaceutically active or acceptable salt forms.

Vitamin A, or a pharmaceutically active form of Vitamin A, is used in the present invention to normalize keratinoid maturation, proliferation and turnover of skin cells. Keratinoid maturation involves keratinocytes, cells that line the follicle under the skin surface. Keratinocytes are involved in the production of acne when these cells, along with the sebum normally produced by the follicle and the hair lining the follicle cause the follicle to become plugged, creating a favorable environment for the proliferation of P. acnes, the bacteria believed to be responsible for formation of skin lesions and pimples. By normalizing keratinoid maturation, as well as proliferation and turnover of skin cells, the follicle is less likely to become plugged. This in turn reduces the occurrence of lesions and pimples, and reduces symptoms related to acne vulgaris such as blemishes, irritation and redness of the skin. Vitamin E, or a pharmaceutically active source of Vitamin E, is present in the invention and acts as an anti-oxidant, thereby protecting the skin cells from the damage caused by oxidation and the free radicals that are produced through the process of oxidation. Vitamin E also acts as an anti-inflammatory and can reduce the inflammation associated with acne. The combination of Vitamins A and E, or pharmaceutically active sources of Vitamins A and E, prevents the formation of milia and comedones, thus depriving growth of the P. acnes bacteria.

Vitamin B₆, or a pharmaceutically active form of Vitamin B₆, in the present invention may facilitate normal testosterone metabolism. A hormonal imbalance plays a role in the production of acne and associated symptoms. Women often experience acne at certain points during their menstrual cycle due to hormone fluctuation. By normalizing testosterone metabolism, Vitamin B₆ may assist in the prevention of pre-menstrual acne flare-ups by normalizing levels of hormones physiologically perturbed by the menstrual cycle.

It has been hypothesized that acne vulgaris is linked to Coenzyme-A due to its activity in fatty acid metabolism and sex hormone synthesis (Lit-Hung Leung, M.D., Pantothenic Acid in the Treatment of Acne Vulgaris, “A Medical Hypothesis”, Journal of Orthromolecular Medicine Vol. 12, No. 2, (1997)). Coenzyme-A is formed from adenosine triphosphate, cysteine and Pantothenic Acid. A deficiency in Pantothenic Acid potentially inhibits or reduces the activity of Coenzyme-A in its fatty acid metabolism and sex hormone synthesis. If the fatty acid metabolism activity is diminished, lipids, which are a combination of fatty acids, begin to accumulate in the sebaceous glands thereby increasing the sebum excretion and in turn, the production of acne vulgaris. Pantothenic Acid is essential for the proper functioning of Coenzyme-A and its related activity; if a deficiency in Pantothenic Acid exists, an increase of sebum production occurs due to the accumulation of lipids in the sebaceous glands. Pantothenic Acid in the present invention therefore reduces sebum production.

Zinc helps promote normal immune functioning. In the treatment of acne, Zinc acts in wound healing and assists oil gland and hormone synthesis.

Selenium also acts as an anti-oxidant, thereby protecting the skin cells from the damage caused by oxidation and the free radicals that are produced through the process of oxidation. Selenium is also active as an anti-inflammatory and acts to reduce the inflammation associated with acne.

Chromium is related to blood sugar. It helps to regulate glucose levels in the blood. It has been found that persons with unstable blood sugar levels have a high occurrence of severe acne (Med Hypotheses, July 1984, 14(3)). By regulating blood sugar levels, chromium acts to reduce acne.

Berberis aquifolium (Oregon grape root), Berberis vulgaris (barberry), Hydrastis Canadensis (goldenseal), or other herbal source of the compound berberine and other isoquinoline alkaloids are in the present invention in a therapeutically effective amount such to inhibit the growth of P. acnes and the lipogensis of sebum production, thereby reducing or eliminating several mechanisms of action in the pathogenesis of acne and related symptoms. Berberine is one of several isoquinoline alkaloids found in plants native to North America. Alkaloids are nitrogenous organic molecules that have pharmacological effects on humans and other animals. Alkaloids are found as secondary metabolites in plants, animals and fungi and can be extracted from their sources by treatment with acids.

In one preferred embodiment, the composition contains a variety of vitamins, minerals and an herbal component, the formulation comprising at least one or more of the following ingredients:
Vitamin A;
Vitamin E;
Vitamin B₆;
Pantothenic Acid;
Zinc;
Selenium;
Chromium;
a source of the compound berberine.

In a further preferred embodiment, the herbal source of the compound berberine is from the group consisting of Berberis aquifolium, Berberis vulgaris and Hydrastis Canadensis.

In a further preferred embodiment, the composition contains a variety of vitamins, minerals, and an herbal component, the formulation comprising at least one or more of the following ingredients in amounts which represent a daily dose administration of the composition as follows:
Vitamin A from approximately 5000 to 10,000 IU;
Vitamin E from approximately 400 to 800 IU;
Vitamin B₆ from approximately 25 to 100 mg;
Pantothenic Acid from approximately 100 to 1000 mg;
Zinc from approximately 30 to 200 mg;
Selenium from approximately 50 to 200 μg;
Chromium from approximately 50 to 200 μg; and
an effective amount of a source of the compound berberine.

In a further preferred embodiment, the composition contains a variety of vitamins, minerals, and an herbal component, the formulation comprising at least one or more of the following ingredients in amounts which represents a single dose of the composition as follows:
Vitamin A from approximately 1500 to 3500 IU;
Vitamin E from approximately 100 to 300 IU;
Vitamin B₆ from approximately 5 to 35 mg;
Pantothenic Acid from approximately 30 to 350 mg;
Zinc from approximately 10 to 70 mg;
Selenium from approximately 15 to 70 μg;
Chromium from approximately 15 to 70 μg; and
an effective amount of a source of the compound berberine.

In one preferred embodiment, the composition is suitable for oral administration, although any suitable route of administration may be used in a therapeutically effective dosage of the composition. Other routes of administration include, for example, parenteral, intravenous, topical or other like forms of administration.

For oral administration, the dosage form of the composition includes tablets, capsules, gel caps, caplets or other suitable forms of oral administration. Suitable dosage forms for other routes of administration include solutions, liquids, suppositories or other suitable forms for administration other than oral administration.

The nutritional product, dietary supplement or pharmaceutical composition used in the present invention includes the active ingredients as described above and may contain other inactive ingredients, pharmaceutically active carriers, and excipients. The nutritional product, dietary supplement or pharmaceutical composition may optionally include other therapeutic ingredients.

### EXAMPLE 1

A clinical study was performed to determine the effectiveness of the present invention for the treatment of acne vulgaris and related skin disorders. The study was conducted using twelve subjects from the patient population of a general dermatology outpatient practice. The study consisted of pre-treatment and post-treatment subjective (patient) and objective (physician) evaluations.

Patients were instructed to take 3 tablets daily, preferably one before each meal. The patients were evaluated before taking the supplement, after one month of supplementation and after two months of supplementation. Patients were instructed to use mild, non-comodeogenic products during the study. Patients were not allowed to use other methods of treatment for their skin condition or antibiotics for other conditions.

At the conclusion of the study, three patients were lost to follow-up, one patient dropped out of the study to receive conventional treatment and one patient moved to another state and was unable to follow up. Seven subjects fully completed the study and the data presented here represents those subjects who had the full two-month course of the composition.

Pre-Treatment

Prior to initiation of the study, patients completed a pre-treatment Acne Quality of Life (AQL) questionnaire. The questionnaire consisted of nine questions regarding any negative effects of their acne on their quality of life which the subject scored as 0-3 or N/A per question with 0 being no impact, 3 being very significant and N/A as not applicable. Patients also completed a pre-treatment Likert Scale of Satisfaction (LSS) rating their level of satisfaction with their prior treatment on a scale of 0-10 with 0 being totally dissatisfied and 10 being totally satisfied. The physician completed a pre-treatment Global Acne Assessment Scale (GAAS) for each subject, giving a quantitative score based upon the number and severity of acne lesion using a scale of 0-5 with 0 being normal skin and 5 being highly inflammatory acne. Individual lesions of both inflammatory and non-inflammatory type were counted and logged.

Pre-Treatment Results

The pre-treatment severity of negative impact of the subjects' acne on nine different indicators of their quality of life as measured by the AQL on a scale of 0-3 per question averaged 6.4.

The subjects' satisfaction with their previous acne treatments as measured by the LSS on a scale of 0-10 averaged 2.3.

The mean lesion count as measured by the physician was 39.6 for non-inflammatory lesions and 25.9 for inflammatory lesions. The mean total lesion count was 65.0.

The physician's objective assessment of the severity of acne as measured by the GAAS on a scale of 0-5 averaged 3.7 pre-treatment.

Post-Treatment

After the study was completed, patients completed a post-treatment Acne Quality of Life (AQL) questionnaire. The questionnaire consisted of nine questions regarding any negative effects of their acne on their quality of life which the subject scored as 0-3 or N/A per question with 0 being no impact, 3 being very significant impact and N/A as not applicable. Patients also completed a post-treatment Likert Scale of Satisfaction (LSS) rating their level of satisfaction with their prior treatment on a scale of 0-10 with 0 being totally dissatisfied and 10 being totally satisfied. The physician completed a post-treatment Global Acne Assessment System (GAAS) for each subject, giving a quantitative score based upon the number and severity of acne lesions using a scale of 0-5 with 0 being normal skin and 5 being highly inflammatory acne. Individual lesions of both inflammatory and non-inflammatory type were counted and logged. The physician also completed a Physician Global Assessment (PGA) of the overall degree of improvement seen quantified on a scale of 0-4 with 0 being worsening and 4 being marked improvement.

Post-Treatment Results

The severity of negative impact of the subjects' acne on nine different indicators of their quality of life as measured by the AQL on a scale of 0-3 averaged 1.0 post-treatment, as compared to 6.4 pre-treatment, resulting in an 84% reduction in the impact of their acne of their quality of life.

The subjects' satisfaction with their treatment during this study as measured by the LSS on a scale of 1-10 averaged 7.9, as compared to their satisfaction with prior treatments of 2.3, resulting in a greater than 70% increase.

The number of non-inflammatory lesions as measured by the physician post-treatment was 20.6, resulting in a 48% decrease.

The number of inflammatory lesions as measured by the physician post-treatment was 11.4, resulting in a 56% decrease.

The number of total lesions as measured by the physician post-treatment was 32.0, resulting in a 51% decrease.

The physician objective assessment of the severity of acne as measured by the GAAS scale averaged 1.9 post-treatment, as compared to an average GAAS pre-treatment score of 3.7, resulting in an improvement of 49%.

The physician's subjective assessment of the severity of acne after treatment on the Physician Global Assessment was 3.0, indicating moderate overall improvement as a group.

TABLE 1
Acne Quality of Life Survey

| Patient Number | Pre-Treatment | Post-Treatment |
|---|---|---|
| 1 | 2 | 2 |
| 2 | 14 | 2 |
| 3 | 2 | 1 |
| 4 | 6 | 0 |
| 5 | 4 | 1 |
| 6 | 12 | 0 |
| 7 | 5 | 1 |
| Mean | 6.4 | 1.0 |

% Difference Between Pre- and Post-Treatment 84%

TABLE 2
Likert Scale of Satisfaction

| Patient Number | Pre-Treatment | Post-Treatment |
|---|---|---|
| 1 | 2 | 2 |
| 2 | 3 | 8 |
| 3 | 5 | 10 |
| 4 | 2 | 9 |
| 5 | 3 | 7 |
| 6 | 0 | 10 |
| 7 | 1 | 9 |
| Mean | 2.3 | 7.9 |

% Difference Between Pre- and Post-Treatment 71%

TABLE 3
Non-inflammatory Lesion Counts

| | Pre-Treatment | Mid-Treatment | Post-Treatment |
|---|---|---|---|
| Patient Number | (Pre) | (Mid) | (Post) |
| 1 | 54 | 44 | 42 |
| 2 | 44 | 23 | 10 |
| 3 | 64 | 17 | 34 |
| 4 | 72 | 35 | 23 |
| 5 | 17 | 24 | 12 |
| 6 | 13 | 15 | 12 |
| 7 | 13 | 3 | 11 |
| Mean | 39.6 | 23.0 | 20.6 |

% Difference Between Pre- and Mid-Treatment 42%
% Difference Between Pre- and Post-Treatment 48%

TABLE 4
Inflammatory Lesion Counts

| | Pre-Treatment | Mid-Treatment | Post-Treatment |
|---|---|---|---|
| Patient Number | (Pre) | (Mid) | (Post) |
| 1 | 29 | 31 | 20 |
| 2 | 15 | 7 | 3 |
| 3 | 29 | 14 | 14 |
| 4 | 16 | 8 | 12 |
| 5 | 46 | 3 | 20 |
| 6 | 31 | 12 | 5 |
| 7 | 15 | 3 | 6 |
| Mean | 25.9 | 11.1 | 11.4 |

% Difference Between Pre- and Mid-Treatment 57%
% Difference Between Pre- and Post-Treatment 56%

TABLE 5
Total Lesion Count

| | Pre-Treatment | Mid-Treatment | Post-Treatment |
|---|---|---|---|
| Patient Number | (Pre) | (Mid) | (Post) |
| 1 | 83 | 75 | 62 |
| 2 | 59 | 30 | 13 |
| 3 | 93 | 31 | 48 |
| 4 | 88 | 43 | 35 |
| 5 | 63 | 27 | 32 |
| 6 | 44 | 27 | 17 |
| 7 | 25 | 6 | 17 |
| Mean | 65.0 | 34.1 | 32.0 |

% Difference Between Pre- and Mid-Treatment 48%
% Difference Between Pre- and Post-Treatment 51%

TABLE 6
Global Acne Assessment Scale

| | Pre-Treatment | Mid-Treatment | Post-Treatment |
|---|---|---|---|
| Patient Number | (Pre) | (Mid) | (Post) |
| 1 | 3 | 3 | 2 |
| 2 | 4 | 3 | 1 |
| 3 | 4 | 3 | 3 |
| 4 | 3 | 2 | 2 |
| 5 | 4 | 1 | 2 |
| 6 | 4 | 2 | 1 |
| 7 | 4 | 1 | 2 |
| Mean | 3.7 | 2.1 | 1.9 |

% Difference Between Pre- and Mid-Treatment 43%
% Difference Between Pre- and Post-Treatment 49%

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

1. A composition for use in a nutritional product, dietary supplement or pharmaceutical composition comprising:
at least one antioxidant component in an amount sufficient to protect against damage caused by oxidation and to reduce free radicals produced in the process of oxidation;
at least one anti-inflammatory component in an amount sufficient to reduce inflammation;
at least one immune modulating component in an amount sufficient to facilitate the immune system response for the repair and regeneration of damaged skin cells;
at least one component which normalizes keratinization;
at least one hormone regulating component in an amount sufficient to normalize hormonal fluctuations;
at least one component which reduces the production of sebum; and
at least one herbal compound that is a source of berberine.

2. The composition of claim 1 wherein the at least one herbal compound that is the source of berberine is Berberis aquifolium,

3. The composition of claim 1 wherein the at least one herbal compound that is the source of berberine is Berberis vulgaris.

4. The composition of claim 1 wherein the at least one herbal compound that is the source of berberine is Hydrastis Canadensis.

5. The composition of claim 1 wherein the at least one antioxidant component is Vitamin E or any pharmaceutically active source thereof.

6. The composition of claim 1 wherein the at least one antioxidant component is Selenium or any active salt thereof.

7. The composition of claim 1 wherein the at least one anti-inflammatory component is Vitamin E or any pharmaceutically active source thereof.

8. The composition of claim 1 wherein the at least one anti-inflammatory component is Selenium or any active salt thereof.

9. The composition of claim 1 wherein the at least one immune modulating component is Zinc or any active salt thereof.

10. The composition of claim 1 wherein the at least one component which normalizes keratinizaton is Vitamin A or any pharmaceutically active source thereof.

11. The composition of claim 1 wherein the at least one hormone regulating component is Vitamin B₆ or any pharmaceutically active source thereof.

12. The composition of claim 1 wherein the at least one component which reduces the production of sebum is Pantothenic Acid.

13. The composition of claim 1 wherein the at least one glucose modulating component is Chromium or any active salt thereof.

14. A method for the treatment of acne and related skin disorders which comprises administering:
at least one antioxidant component in an amount sufficient to protect against damage caused by oxidation and to reduce free radicals produced in the process of oxidation;
at least one anti-inflammatory component in an amount sufficient to reduce inflammation;
at least one immune modulating component in an amount sufficient to facilitate the immune system response for the repair and regeneration of damaged skin cells;
at least one component which normalizes keratinization;
at least one hormone regulating component in an amount sufficient to normalize hormonal fluctuations;
at least one component which reduces the production of sebum; and
at least one herbal compound that is a source of berberine.

15. The method of claim 14 wherein the administration is oral.

16. The method of claim 14 wherein the composition is administered in conjunction with at least one additional nutritional product, dietary supplement or pharmaceutical composition used to treat acne, related skin disorders of said acne and said related skin disorders.
